(19) United States
(12) Patent Application Publication
(10) Pub. No.: US 2007/0293753 A1
(43) Pub. Date: Dec. 20, 2007

(54) Radiometric Approach to Temperature Monitoring Using a Magnetic Resonance Scanner

(76) Inventors: Abdel-Monem El-Sharkawy, Baltimore, MD (US); Ergin Atalar, Bilkent Ankara (TR)

Correspondence Address: MCKENNA LONG & ALDRIDGE LLP, 1900 K STREET, NW, WASHINGTON, DC, 20006, US

(21) Appl. No.: 10/585,570

(22) PCT Filed: Jul. 7, 2004
(86) PCT No.: PCT/US04/21642

§ 371 Date: Mar. 21, 2007

(51) Int. Cl. A61B 5/05

(52) U.S. Cl. 600/412

(57) ABSTRACT

Disclosed is a method and system for acquiring absolute temperature imagery using an MR scanner. The method involves using the RF coil as a passive antenna, and performing radiometric measurements of the noise variance of the target within the field of view of the RF coil. The noise variance corresponds to the absolute temperature of the volume within the field of view of the RF coil. The room of the MR scanner is used for electromagnetic shielding during the acquisition of radiometric data. This method may be performed with minimal or no add-ons to existing MR scanner hardware. Disclosed are a method for calibrating an MR scanner for radiometric temperature measurements, and a method for acquiring and generating thermal imagery with a calibrated MR scanner.

This application claims the benefit of U.S. Provisional Patent Application No. 60/485,299, filed on Jul. 7, 2003, which is hereby incorporated by reference for all purposes as if fully set forth herein.

Research and development efforts associated with the subject matter of this patent application was supported by the National Institutes of Health under Grant Nos. R01HL61672 and R01HL57483.

### BACKGROUND OF THE INVENTION

1. Field of the Invention

The present invention relates to the creation of three-dimensional thermal images using a Magnetic Resonance (MR) imaging system, where the imagery shows absolute temperatures within a target tissue.

Three dimensional imaging of absolute temperature within a target tissue provides great benefit in both medical diagnostics and treatment. In medical diagnostics, it is known that tumors, for example, maintain a different temperature relative to the surrounding tissue. This is particularly true with breast cancer. Thus, thermal imagery of the absolute temperature would assist greatly in diagnosing tumors. Another example of medical diagnosis that might benefit from thermal imagery is the detection of inflamation associated with, for example, liver disease or atherosclerosis.

In the field of medical treatment, thermal therapy is, for example, a procedure in which a concentrated thermal dose is delivered to a tumor to shrink ,and/or eliminate the tumor. Thermal imagery of the absolute temperature of both the tumor and the surrounding tissue during thermal therapy would help ensure that sufficient thermal energy is being imparted to the tumor, while thermal damage to the surrounding tissue may be assessed and/or mitigated.

Thermal imagery might also be beneficial in monitoring specific absorption rate (SAR) during MR procedures. This is particularly important for maintaining the safety of the patient. This may include interventional coils and high field imaging.

2. Discussion of the Related Art

Passive microwave radiometry has long been used in astronomy and remote sensing of Earth surface temperatures, often from airborne or spaceborne platforms. Such systems typically use passive radiometers operating in the 1-300GHz range, at multiple frequencies. These systems generally exploit the spectral characteristics of the received multispectral microwave energy to derive temperature data corresponding to the surface. However, measurements in the lower frequency (i.e. longer wavelength) regions of the electromagnetic spectrum, such as in the radio frequency (RF) region of the spectrum, can be difficult due to the weakness of the signal relative to system thermal noise and interference from the environment.

The passive (i.e., non-invasive) nature of temperature microwave remote sensing makes it a particularly attractive solution for medical imaging applications. However, the problems associated with system noise and RF environmental factors, such as external noise factors, make the use of such temperature measurement techniques difficult in medical applications. For example, a medical facility presents many RF noise sources that are not encountered by a remote sensing instrument in the depths of space. Given the weakness of the RF signal being measured, and the noise inherent in a medical environment, any RF measurements will require extremely robust shielding, and high quality factor components.

The benefits of non-invasive absolute temperature imagery, and the inherent difficulties incurred in taking such measurements, underscore the need to take very sensitive RF measurements in a noisy environment, such as an MR environment, in order to accurately generate thermal images reflecting the temperature inside the object being scanned.

### SUMMARY OF THE INVENTION

Accordingly, the present invention is directed to a radiometric approach to temperature monitoring using a magnetic resonance scanner that substantially obviates one or more of the problems due to limitations and disadvantages of the related art.

An advantage of the present invention is to provide more accurate, absolute non-invasive thermal imaging of a target tissue volume.

Another advantage of the present invention is to provide more effective diagnoses of medical ailments such as the identification of tumors.

Another advantage of the present invention is to provide real time thermal imagery to better assist in the treatment of patients.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and described, a system for generating thermal imagery using an MR scanner comprises: an RF coil; a tuning means connected to the RF coil; a pre-amp connected to the tuning means; a demodulator connected to the output of the pre-amp; a digitizer connected to the output of the demodulator; and a computer connected to the output of the digitizer, the computer having a computer readable medium encoded with a program for collecting noise signals detected by the RF coil, calculating a variance of the noise signals, and converting the variance to a temperature.

In another aspect of the present invention, a method for using an MR scanner to measure absolute temperature of a target volume, the method comprises the steps of: tuning an RF coil; collecting a plurality of signal data from the RF coil; determining a variance corresponding to the plurality of signal data; and converting the variance to an absolute temperature data.

In another aspect of the present invention, a method for calibrating an MR scanner for measuring absolute temperature of a target volume, the method comprises the steps of: placing a first phantom having a first temperature within a field of view of an RF coil; tuning the RF coil; collecting a first plurality of signal data from the RF coil; determining a first variance corresponding to the first plurality of signal data; placing a second phantom having a second temperature within the field of view of the RF coil; collecting a second plurality of signal data from the RF coil; determining a second variance corresponding to the second plurality of signal data; and computing a calibration coefficient corresponding to the relation between the first temperature and the first variance, and the second temperature and the second variance.

In another aspect of the present invention, a computer readable medium encoded with a program comprising the steps of: issuing an instruction to tune an RF coil; collecting a plurality of signal data from the RF coil; determining a variance corresponding to the plurality of signal data; and converting the variance to an absolute temperature data.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 shows an exemplary MR scanner system to be used for performing radiometric temperature measurements according to the present invention;

FIG. 2 shows an exemplary configuration of RF coils, with their respective gain patterns defining a field of view;

FIG. 3 depicts a process for performing radiometric temperature measurements according to the present invention;

FIG. 4a shows exemplary raw voltage data from two RF coils;

FIG. 4b shows histograms of raw noise voltages collected from two RF coils, showing the variance used for computing temperature;

FIG. 4c shows multiple histograms of voltage data, revealing a variance of the variance of the measured noise;

FIG. 5 shows an exemplary process for calibrating the MR scanner for performing radiometric temperature measurements according to the present invention;

FIG. 6 is an exemplary phantom that may be used as a calibration reference for performing absolute temperature measurements; and

FIG. 7 shows an alternate exemplary process for generating absolute thermal imagery in accordance with the present invention.

### DETAILED DESCRIPTION OF THE ILLUSTRATED EMBODIMENTS

The present invention uses an MR scanner as a passive RF radiometer to detect thermal noise radiated from a target tissue. It is known that tissue (or any material, for that matter) emits electromagnetic radiation that varies with temperature. In the RF region of the electromagnetic spectrum, the statistical variance of the noise present in the RF radiated power is a function of temperature of the tissue. In an MR scanner, the RF noise that is generated by the tissue in the scanner may induce a current in the RF coil. The induced current in the RF coil may be detected by the electronics of the MR scanner, and processed to produce measurement of the absolute temperature of the tissue. The thermal noise generated by the tissue is a low amplitude signal, and subject to environmental noise and interference. However, by using the MR scanner room for electromagnetic shielding, one may create a sufficiently quiet RF environment around the tissue, enabling accurate measurement of the statistical variance of the thermal noise. Given the ability to steer the field of view of the RF coil, one may generate an image of absolute temperature of a tissue by iteratively acquiring temperature measurements and scanning the field of view.

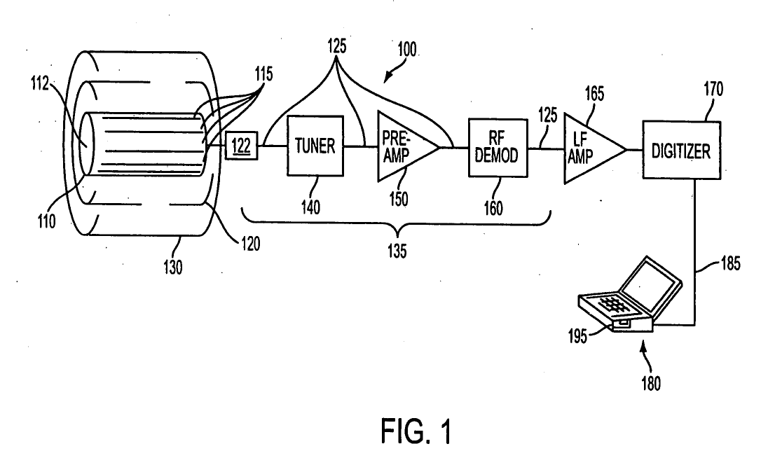

FIG. 1 shows an MR scanner 100 for performing radiometric thermal imaging according to an exemplary embodiment of the present invention. The MR scanner 100 may employ known or existing hardware and systems, such as, for example, hardware associated with a GE Signa™ 1.5 Tesla scanner system. The MR scanner 100 may comprise a main magnet 130; a gradient magnet 120; an RF coil 110 or alternatively, a plurality of individual RF coils 115; a sample chamber 112, in which a target is placed for measurement; a commutator 122; RF cabling 125; a tuner 140; a pre-amp 150; an RF demodulator 160; a low frequency amplifier 165; a digitizer 170; data cabling 185; and a data system 180. The following descriptions of the MR scanner 100 assume the use of multiple RF coils 115, although it will be apparent to one skilled in the art that the following descriptions would equally apply to a scanner 100 with a single coil 110. The room housing the MR scanner 100 typically includes electromagnetic shielding, to isolate the MR scanner 100 from the RF environment.

The MR scanner 100 generally comprises a gradient magnet 120, and a main magnet 130. In the nominal operating mode of an MR scanner, magnets 120 and 130 are passive, and the MR scanner 100 room shielding mitigates RF environmental interferences that would corrupt measurements of the thermal noise radiated by the target tissue. The scanner room passively serves as a Faraday cage, and substantially enables the RF coils 115 to detect the low amplitude RF noise associated with the temperature of the target tissue. While the magnets 120 and 130 may be operating, there should be no RF transmission when the system is detecting the thermal RF noise.

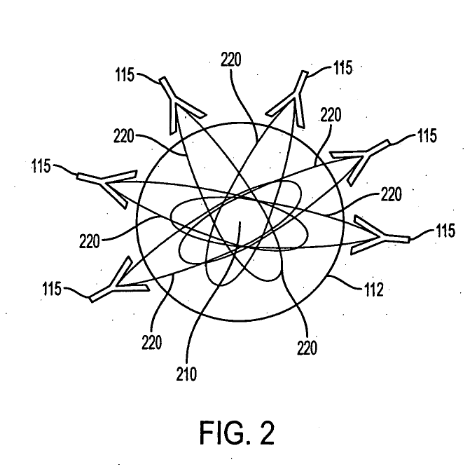

In a preferred embodiment of the present invention, the RF coils 115 serve as a passive antennas, detecting RF noise power radiated by the target tissue. FIG. 2 shows an exemplary configuration of RF coils 115 positioned around the sample chamber 112. As shown, each RF coil 115 has an individual gain pattern 220, which may be superimposed to define a field of view 210, which corresponds to a volume pixel, or voxel. As mentioned earlier, thermally generated RF noise power that is radiated within the gain pattern 220 of a given RF coil 115 induces a current in the RF coil 115. Accordingly, generated RF noise power radiated within the field of view 210, induces a distinct current in each of the RF coils 115.

The field of view 210 may be steered, enabling the RF coils 115 to scan throughout the target tissue. The field of view 210 may be steered by a variety of mechanisms. In a preferred embodiment, the field of view 210 may be scanned via a technique like that used in electrical impedance tomography, incorporating phased array coils or multiple coils placed side by side. Phased array coils with 16 elements, and 64 channel systems, are known and commercially available. Another approach is to mechanically scan with each RF coil 115. Other approaches for scanning the field of view 210 are possible. If a single RF coil 110 is used, the field of view may be steered by successively acquiring data at different frequencies, as it is known that an RF coil gain pattern has a distinct shape that is a function of frequency. Thus, by changing the frequency, and identifying the changes in signal amplitude, it may be possible to determine the location of a given voxel. Another method for steering the field of view would be to use different coil modes, such as using the modes for a birdcage coil. Still further, scanning for the purpose of generating either a two-dimensional or a three-dimensional thermal image is consistent with the present invention.

RF cables 125 guide the signal to and from each of components 115, 112, 122, 140, 150, 160 and 170, which make up an RF signal path 135. The RF cables 125 preferably include high quality factor and low noise components, and robust shielding, as would be appropriate for the detection and measurement of faint signals. Such cabling is typically provided in existing MR scanners.

In a preferred embodiment, a commutator 122 commutates the signals from each of the RF coils 115 into a single RF signal path 135. The advantages of commutation include the need for one set of cables 125, one pre-amp 150, and one demodulator 160. Decommutation may occur at one of many points within the RF signal path 135, at the digitizer, or in the data system 180. In an alternate embodiment, no commutation is done, and each RF coil 115 may have its own RF signal path 135.

The tuner 140 matches the impedance between the RF coil 115, the RF cabling 125, and the pre-amp 150. Impedance matching is important to prevent attenuation of the signal from the RF coil 115, which may already be a low amplitude signal. The tuner 140 may be integral to the MR scanner 100, or may be added to the scanner 100 by being connected to the signal path between the RF coil 115 and the pre-amp 150. One skilled in the art will be able to connect the tuner 140 to the MR scanner 100 as shown in FIG. 1.

In a preferred embodiment, the tuner 140 may include the following: a network analyzer, or equivalent, which measures the impedance characteristics of the signal path from the RF coil 115 to the pre-amp 150; a controller, such as a processor or microcontroller; and a variable capacitor, or similar component for setting the impedance. The variable capacitor may be mechanically or electronically controlled. The preferred embodiment of the tuner 140 operates automatically as a closed loop control system. Alternatively, the tuner 140 may include a network analyzer, or similar; and a plurality of non-magnetic capacitors. In this embodiment of the tuner 140, an operator monitors the readout of the network analyzer and manually adjusts the capacitors in order to provide impedance matching. It will be apparent to one skilled in the art that other tuner implementations are possible with the present invention.

The pre-amp 150 amplifies the signal from the RF coils 115 to an amplitude appropriate for the dynamic range of the digitizer 170. In a preferred embodiment, the pre-amp 150 has a substantially linear gain throughout its dynamic range, and includes high quality factor and low noise characteristics, as would be necessary to accurately and precisely amplify a low amplitude signal. Existing MR scanners typically employ a pre-amp 150 with these characteristics.

The RF demodulator 160 converts the amplified RF noise signal detected by the RF coils 115 to a baseband signal. In doing this, the RF demodulator 160 shifts the center frequency of the RF signal to DC. The RF demodulator 160 may be the same or similar to those found in existing MR scanners, and preferably includes high quality components and construction. In a preferred embodiment, the RF demodulator 160 includes at least one local oscillator and appropriate components for applying a distinct phase to the local oscillator signal applied to the amplified signal from each RF coil 11 5. Existing MR scanners typically employ phase sensitive detectors, quadrature detectors, and the like, that may substantially serve the purpose of the demodulator 160 of the preferred embodiment.

The digitizer 170 converts the baseband signal from the RF demodulator 160 into digital data. As one skilled in the art will readily appreciate, the digital data includes a sequence of digital words representing samples of the baseband signal, sampled at a configurable rate. The digital data may represent the baseband signal in a complex format, such as an in-phase and quadrature format. Both the in-phase and the quandrature channels may be used for estimation, as demodulation does not affect the flat spectrum of the noise. The digital data may also include ancillary data such as time tags, and bits representing which RF coil 115 is the source of the signal. In a preferred embodiment, which employs a commutator 122, the digitizer 170 may provide digital data from all of the RF coils 115 in a single multiplexed data stream, or it may provide separate digital data streams, one per RF coil 115. The digitizer 170 transmits the digital data to the data system 180 via a digital data cable 185, which may comprise a single conductor, or multiple conductors, as is found in a ribbon cable. The digital data cable 185 may include a network and/or a wireless link.

The data system 180 performs functions that may include the following: controlling MR scanner 100 components; acquiring digital data from the digitizer 170; processing digital data to generate images; communicating with remote operators and databases; and performing diagnostics on the components within the MR scanner 100. The data system 180 may include an architecture that comprises a single computer, multiple computers, a combination of standalone computers and embedded processors, or a combination of a local computers and remote computers connected by a network. It will be apparent to one skilled in the art that many other data system 180 architectures may be possible in the present invention. The data system 180 stores and runs the software 195, which performs much of the functionality of the present invention.

The software 195 may be stored and executed on one or more of the computers making up the data system 180, according to at least any of the data system 180 architectures listed above. The software 195 may include special purpose software for implementing the present invention, and it may include software integral to existing MR scanners. Further, the software 195 may include libraries of software functions dedicated to the MR scanner 100 in which the software 195 is being executed.

The software 195 may include instructions for performing at least any of the following: issuing commands to configure the MR scanner 100; issuing commands to control any of the MR scanner 100 components; issuing commands to acquire digital data from the digitizer 170; converting the acquired digital data into temperature data; generating images of temperature data; presenting images and data to the operator; accepting commands and parameters from the operator; storing image data values; storing configuration parameter values; issuing commands to calibrate the MR scanner 100 for temperature measurements; and communicating with remote computers and databases.

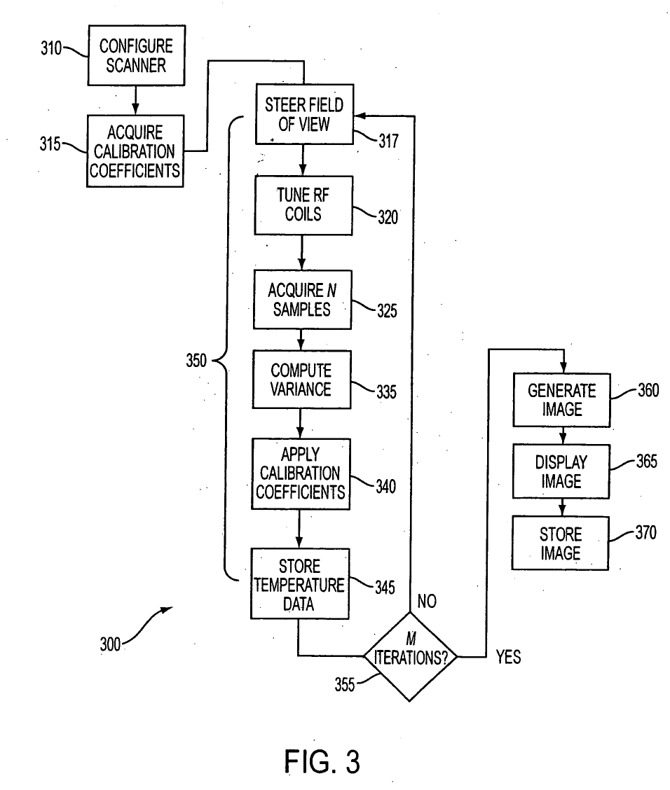

The software 195 includes instructions for generating images of absolute temperature according to the present invention. FIG. 3 shows an exemplary process 300 that may be implemented by software 195 toward that end. The exemplary process 300 may include configuration steps 310 and 315; a data acquisition cycle 350, which includes steps 317-345, and image processing steps 360-370.The process shown in FIG. 3 may be performed in parallel with nominal M scanning operations as known in the related art.

Process 300 may begin with step 310, in which the software 195 executes instructions to configure the MR scanner 100. In step 310, the software 195 performs actions that may include acquiring and setting configuration parameters, and issuing commands to components to prepare for operation. The software 195 acquires values for configuration parameters for operating the MR scanner 100, which may include bandwidth B, number of samples per measurement N, center frequency for the RF demodulator 160, number of scans per image M, and instructions for steering the field of view 210. The software 195 may acquire these values by one or more of the following: by retrieving the values from specific memory locations; by prompting the operator and retrieving the values from keyboard inputs; by issuing instructions to query one or more databases; and the like. Once the software 195 has acquired these parameter values, it may store or buffer them in memory locations allocated to the software 195 by the operating system of the data system 180. Commands issued by the software 195 in step 310 may include instructions to turn off magnets 120 and 130, and instructions to confirm that components such as those in FIG. 1 are operational.

In step 315, the software 195 acquires calibration coefficient values for converting measured thermal noise into temperature. The calibration coefficient values may be generated by the software 195 in a separate calibration procedure described later herein. The software 195 may acquire and buffer the calibration coefficient values by at least any of the methods listed above for acquiring configuration parameter values in step 310. The software 195 may acquire calibration coefficient values that correspond to the configuration parameters acquired in step 310. For instance, the calibration coefficient values required for accurate temperature calculation may depend on the bandwidth B and the number of samples N. However, the Bandwidth B term may not be necessary. Generally, the wider the bandwidth B, the greater the amplitude of the RF signal. Also, it is generally true that the greater the number of samples N, the more accurate the statistical variance measurement.

In step 317, the software 195 may steer the field of view 210 to a given voxel location. For example, the software 195 may do this by using the instructions for steering the field of view 210, acquired in step 310. The instructions may contain RF coil 115 phase offset information corresponding to a given voxel location. In step 317, the software 195 may issue commands to steer the field of view 210 according to at least one of the other steering approaches described earlier, such as electric impedance tomography techniques or mechanical scanning.

In step 320, the software 195 issues a command to tune the RF coils 115. If the tuner 140 has a dedicated controller, as in the preferred embodiment, the software 195 may issue a command to the tuner's 140 controller, instructing it to match the impedance of the RF coils 115. Depending on the design of the tuner 140, the software 195 may implement closed loop control of the tuner 140 otherwise done by the tuner's 140 controller. Alternatively, if the tuner 140 operates manually, the software 195 may display an instruction to the operator to match the impedance of the RF coils 115, and then wait for input (via keyboard, or mouse, for example) from the operator indicating that the impedance is matched. Auto-tuning is inherent in many scanners. In this case, the turning process may be performed in the background.

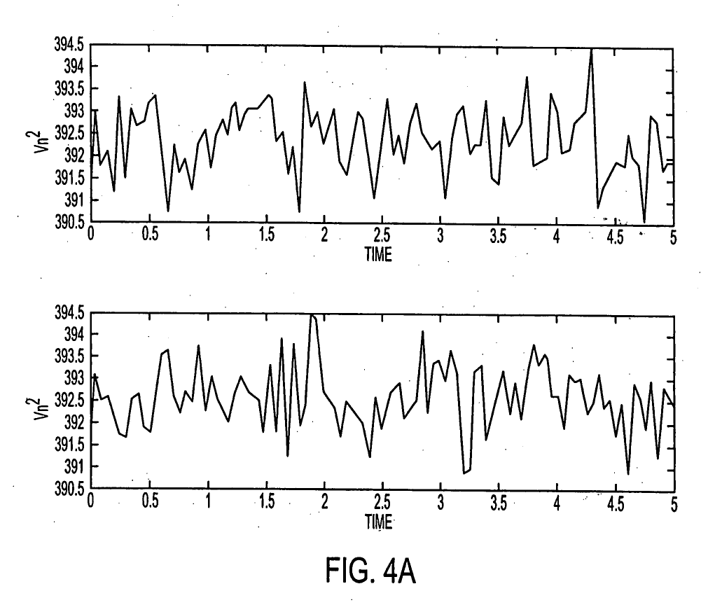

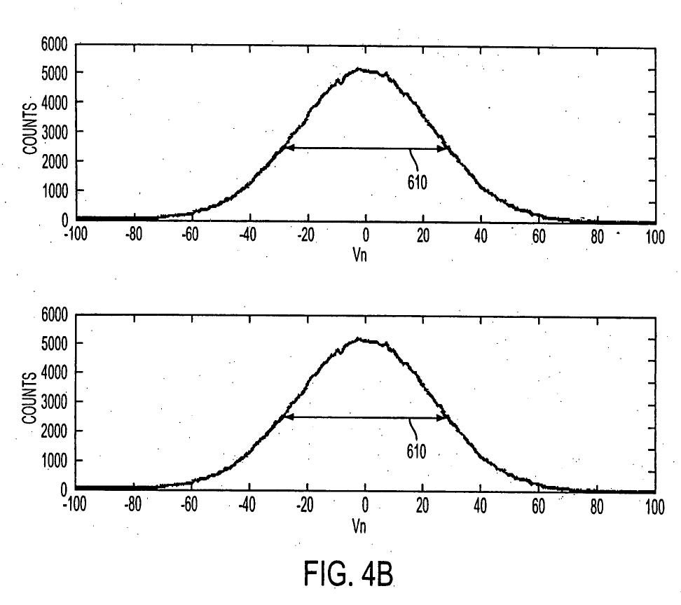

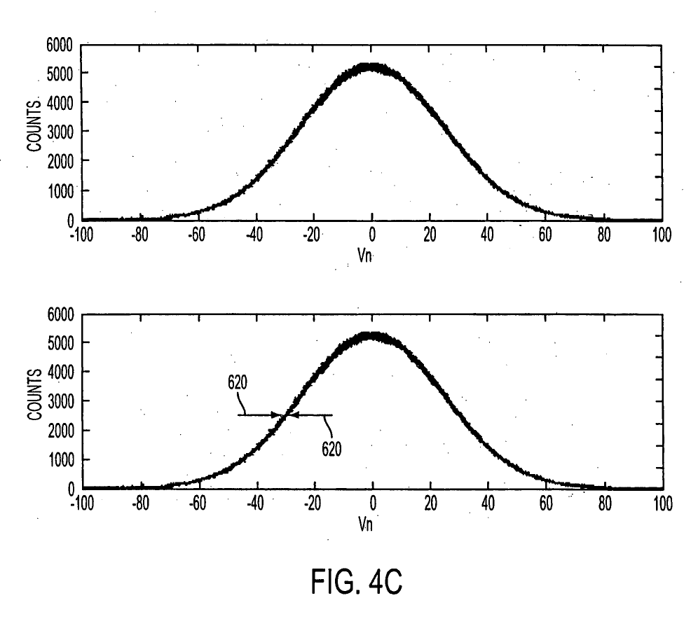

In step 325, the software 195 issues a command to acquire N samples of digital data from the digitizer 170. As discussed above, the digital data comprises digital samples of baseband amplified signals from the RF coils 115 that correspond to current induced by RF noise power radiated by the target tissue. In response to the software's 195 command, the digitizer 170 may transmit the digital data to the data system 180 in a substantially continuous data stream, or may transmit the data in packets. The software 195 may acquire the digital data values, format the data for subsequent processing, and store the formatted digital data values in one or more arrays of memory locations. Formatting the digital data may include segregating the data into distinct memory locations by RF coil. Further, the software 195 may plot the formatted digital data values, as shown in FIG. 4a.

Having acquired, formatted and stored the digital data values, the software 195 may compute the statistical variance of the data as shown in step 335. The software 195 may compute the variance individually per RF coil 115. The software 195 may compute the variance using one or more software routines found in any of several known mathematical libraries. The software 195 may display the computed statistical variance value corresponding to each RF coil's 115 data, and store the variance value in memory. The software 195 may also plot the collected data in the form of a histogram 310, as shown in FIG. 4b. A histogram 310 may provide the operator with useful information regarding the quality of the data collected. The software may also overlay the histograms for each RF coil's 115 data in a single plot, as shown in FIG. 4c. As shown in FIG. 4c, overlaying the histograms provides an indication of the “variance of the variance,” or the precision to which each RF coil 115 is measuring the temperature of the voxel.

In step 340, the software 195 may use the statistical variance value of the data, the calibration coefficient values acquired in step 315, and the configuration parameter values acquired in step 310, to calculate the absolute temperature of the target tissue at the location defined by the superimposed fields of view of the RF coils 115. The software 195 may calculate a value representing the temperature T, as measured by any of the RF coils 115, using the following relation:
Vₙ²=4(1−|Γ|²)G²FkBRT
where Vₙ² is the statistical variance computed in step 335; Γ is the Reflection Coefficient of the RF coil 115; G is the system gain; F is the noise figure of the system; R is the input resistance of the system; k is Boltzman's constant; and B is the bandwidth of the system. Bandwidth B is a configuration parameter acquired by the software 195 in step 310. Parameters G, F, R, and k may be collectively folded into a single calibration coefficient, which corresponds to a given coil 115. The value of the calibration coefficient corresponding to GFkR may be measured, computed, and stored in an exemplary calibration process to be described later.

With the tuner 140 matching the impedance of the RF coil 115, the reflection coefficient r is substantially equal to zero. Further, the gain G and noise figure F may be determined by calibrating and modeling the pre-amp 150.

In step 345, the software 195 stores the computed temperature T value in memory. The software 195 may store the temperature T value along with an index value corresponding to the voxel location, which may be used in later image construction.

00551 With a data acquisition cycle 350 complete, the software 350 determines if M voxels have been processed. The software 195 may do this by incrementing a counter at each acquisition cycle 350 iteration, and comparing the counter value to the value of the configuration parameter M. If the values are equal, the software 195 proceeds to generate an image in step 360. In step 360, the software 195 retrieves the temperature values corresponding to each voxel, and corresponding voxel location information. With this information, the software 195 formats the temperature data into an image format appropriate for displaying on a display device such as a computer monitor, TV, or printer. In step 365, the software transmits the image data, which comprises temperature data and image format information, to an appropriate display device. In step 370, the software stores image data values, along with image format information, on a recording media. The store image step 370 may include writing the image data values to a non-volatile memory on data system 180; transmitting the image data to a remote computer; or transmitting the image data to a database. Methods for two-dimensional and three-dimensional image reconstruction are within the scope of the process described herein.

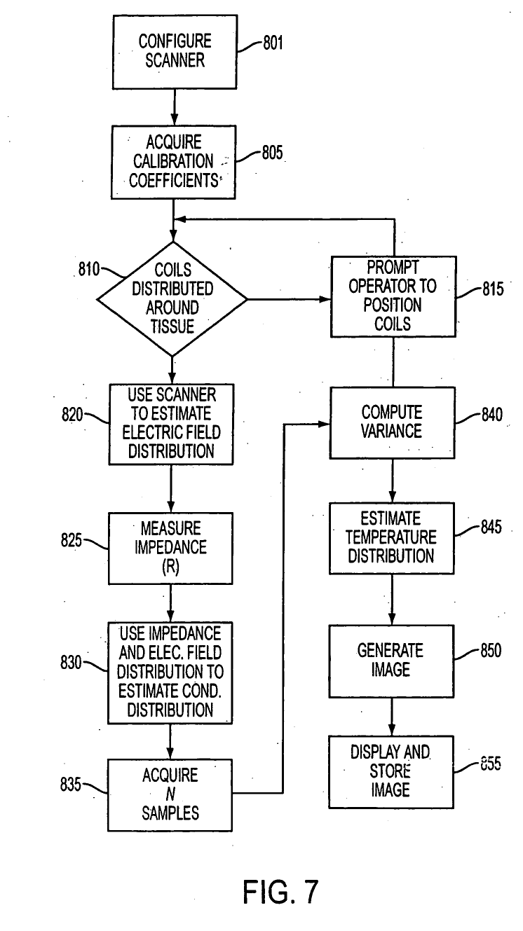

FIG. 7 shows an alternate exemplary process for generating absolute thermal imagery according to the present invention. In this process, imagery is derived by using an approach substantially similar to that used in electrical impedance tomography.

Steps 801 and 805 may be substantially similar to steps 310 and 315 in process 300. In step 810, the software 195 prompts the user to assure that the RF coils 115 are positioned in a distributed fashion around the tissue.

In steps 820, the MR canners 100 measures a spatial distribution of magnetic sensitivity, as is nominally done in MR imagery. The software 195 makes an initial guess at an electric field distribution E(r), computes the corresponding magnetic field distribution B(r), and then computes a projection of B(r), and then computes a projection of B(r) as it would be sensed by the RF coils 115. The software 195 then compares this projection with the magnetic sensitivity distribution measured by the MR scanner 100.

Depending on the comparison between the measured and the estimated magnetic field distribution, the software 195 may then tweak or morph the initial guess at E(r), repeat the estimation of the projected magnetic field, and the comparison of the estimated magnetic field with the measured magnetic sensitivity. This process may repeat until the software 195 converges on an estimation for the electric field distribution that corresponds to the MR measurement.

In step 825, the software 195 measures the impedance R of the RF coil 115. It may do so by injecting a known current, at a specified frequency, through the RF coils 115, and measuring the reflected RF power. Techniques for measuring RF coil impendances are known, and are compatible with the present invention.

Knowing the impedance R, and the electric field distribution E(r), the software 195 may then execute instructions to estimate the electrical conductivity distribution σ(r) using the following relationship.
R=∫σ(r)|E(r)|²dr

Functions called by software 195 may implement numerical methods to estimate σ(r), as described in Ziya Ider, Nevzat G. Gencer, Ergin Atalar, Haluk Tosun, “Electrical Impedance Tomography of Tranlationally Uniform Cylindrical Objects with General Cross Sectional Boundaries,” IEEE Transactions on Medical Imaging, Vol.9, No.1, pp. 49-59 (March 1990.), which is incorporated by reference as if fully disclosed herein. Having converged on an estimate for σ(r), the software 195 stores the appropriate valves.

In steps 835, the software 195 acquires N samples in a manner substantially similar to step 325 in process 300. The software 195 then computes the variance corresponding to the N samples in a manner substantially similar to step 335 in process 300.

In step 845, the software 195 executes instructions to estimate the absolute temperature distribution T(r) in the tissue. The software 195 does so by implementing mathematical functions to estimate T(r) based on the following relationship:

$${V}_{n}^{2} = 4 ( 1 - {\vert Γ \vert}^{2} ) {G}^{2} FkB ( {\int }_{v} T ( r ) σ ( r ) {\vert E ( r ) \vert}^{2} ⅆ r )$$

where 4(1−|Γ|²)G²FkB are calibration coefficients acquired in step 805, E(r) is estimated in step 820, and cy(r) is estimated in step 830. The software 195 may estimate T(r) by a process of reverse integration, according to known numerical methods.

According to the present invention, the calibration coefficients may be derived by properly modeling the gain G and the noise figure F of the pre-amp 150. This may be done without a separate calibration procedure 500. Further, other radiometry techniques, such as those that might employ a Dick Radiometer, may be used for calibration.

Having computed and stored values corresponding to the temperature distribution T(r), the software 195 may generate, display and store temperature image data values in steps 850 and 855, in a manner substantially similar to steps 360, 365 and 370 in process 300.

The temperature measurement techniques in accordance with the exemplary embodiments of the present invention may be combined with other MR thermometry techniques to increase spatial resolution.

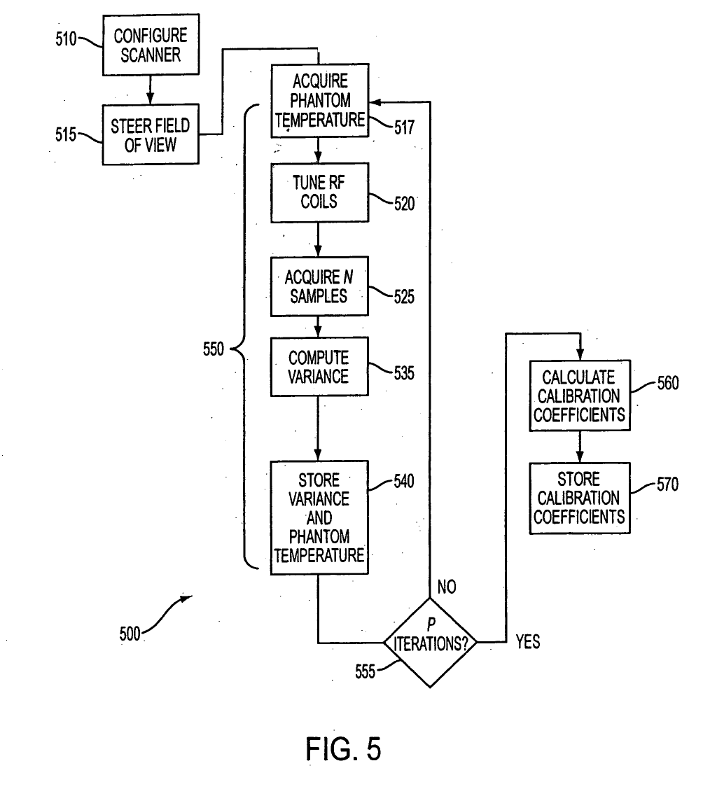

Calibration coefficients may be calculated by calibrating and modelling components of the MR scanner, such as the pre-amp 150, and the digitizer 170. Alternatively, FIG. 5 shows an exemplary calibration process 500 according to the present invention. The exemplary calibration process 500 is similar to process 300, with a few exceptions. For example, in the calibration process 500, the thermally generated RF noise is detected in a phantom, instead of a target tissue.

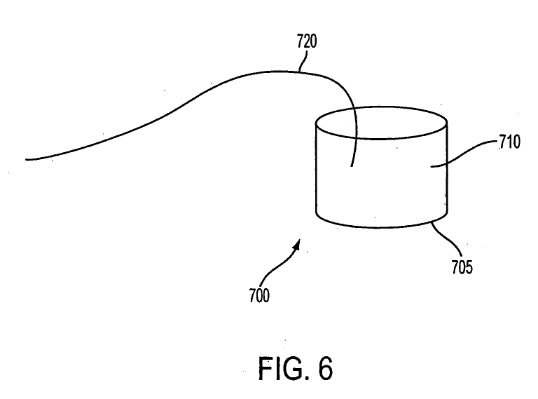

A phantom is a calibration reference with a known temperature, and an electrical conductivity similar to that of human tissue. FIG. 6 shows an exemplary phantom 700, which may include an electrically and thermally non-conductive container 705; a thermally controlled phantom medium 710, which may include material of similar conductivity to the human body; and a temperature sensor 720. The temperature sensor 720 may be a fiberoptic temperature sensor, like that manufactured by FISO, Inc., or similar. A preferred temperature sensor 720 will have substantially zero thermal and electrical conductivity. The temperature sensor 720 may have a transducer, and a data interface through which temperature measurement samples may be acquired by the software 195. The phantom 700 is preferably designed such that it remains thermally stable during one data acquisition cycle 450 of the exemplary calibration process 500.

In calibration process 500, the software issues commands to configure the scanner in step 510 in a substantially similar manner as in step 310 in process 300. In step 515, the software issues commands to steer the field of view 210 such that the resulting voxel remains substantially within the phantom medium 710. Process 500 includes an iterative data acquisition sequence 450, which iterates at least once per phantom 700 temperature, for a total of P iterations. In a preferred embodiment, the software 195 issues commands to acquire the phantom 700 temperature, which may be done by sending commands to query the temperature sensor 720 in phantom 700. Alternatively, the software 195 may display information to the operator, requesting that the operator enter the phantom temperature manually. One of skill in the art will readily recognize that many communication schemes, in which the software 195 sends commands for acquiring the phantom temperature are possible.

Steps 520-535 are substantially the same as steps 320-335 in process 300. In step 540, the software 195 stores the variance computed in step 535, and temperature valves measured by temperature sensor 720 in memory. For each of the P iterations of data acquisition cycle 550, a phantom including a new temperature is used. After P iterations, the software 195 has stored sufficient variance and temperature data values to derive a curve. In a preferred embodiment, P is a configuration parameter.

In step 560, the software 195 executes mathematical functions corresponding to a linear curve fit algorithm, using the variance and temperature values stored in step 540 as input. The software 195 implements the curve fit, such as a linear regression, to calculates a slope. The theoretical slope is given by the equation:
slope=4G²kBR

Where B is the bandwidth of the system; R is the resistance; k is Boltzmann's constant; G is the total system gain; and F is the noise figure, which correspond to the calibration coefficients acquired by the software 195 in step 310 of process 300, and applied to compute temperature in step 340.

The software stores the value corresponding to the slope in step 570. In a preferred embodiment, the software computes and stores a slope value for each RF coil 115, although the slope value may be calculated for one RF coil 115, and stored.

In an alternate embodiment to exemplary calibration process 500, the calibration process may use multiple phantoms 700 each at a different temperature that are placed within the sample chamber 112 at the same time. In this embodiment, the field of view 210 may be scanned once per data acquisition sequence 550 iteration, such that the software 195 may acquire and store the variance and temperature value for each phantom 700 and then steer the field of view 210 to the next phantom in succession.

An alternate way to calibrate the MR scanner 100 for temperature measurements is to use a phantom 700 within which the temperature is changing at a constant rate. In this method, as the temperature of the phantom sweeps through a desired temperature range, the software 195 collects and stores data values from the digitizer 170, along with the phantom's temperature as measured by temperature sensor 720. Once the data is acquired for a given number (i) data acquisition cycles, the software 195 may execute functions that apply regression algorithms to the input data to estimate the changes in the received noise power (ΔP) and the actual measured temperatures (ΔT). The software 195 then may execute instructions to calculate the ratios between these two quantities: rᵢ=ΔPᵢ/ΔTᵢ, where i is the index of the data acquisition cycle. The relative ratios R=rᵢ/rᵢ₊₁ provides a measure of correlation between the noise power and the phantom temperatures measured by temperature sensor 720.

To validate the gain coefficients, the coefficient G may be measured by calibrating the pre-amp 150 and the digitizer 170, and summing the gains. For example, The pre-amp 150 may calibrated by using a noise source, an attenuator, and a noise figure meter. The noise figure meter may be used to measure the total gain and the noise figure of the pre-amp 150. The digitizer 170 may be calibrated using a high precision signal generator and an attenuator. The signal generator may be set to the MR scanner's 100 receiving frequency. The software 195 may record the data from the digitizer 170, and compute the digitizer gain. The digitizer gain may be in units of Volts⁻¹, and represented in dB/Volt. The gain of the digitizer 170 may be added to the gain of the pre-amp 150 to compute the system gain G.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

What is claimed is:

1. A system for generating thermal imagery using an MR scanner comprising:
an RF coil;
a tuning means connected to the RF coil;
a pre-amp connected to the tuning means;
a demodulator connected to the output of the pre-amp;
a digitizer connected to the output of the demodulator; and
a computer connected to the output of the digitizer, the computer having a computer readable medium encoded with a program for collecting noise signals detected by the RF coil, calculating a variance of the noise signals, and converting the variance to a temperature.

2. The system of claim 1, further comprising a means for steering a field of view of the RF coil.

3. The system of claim 1, further comprising:
a main magnet substantially surrounding the RF coil; and
a gradient magnet substantially surrounding the RF coil.

4. The system of claim 1, wherein the tuning means comprises:
a network analyzer; and
a non-magnetic variable capacitor.

5. A method for using an MR scanner to measure absolute temperature of a target volume, the method comprising the steps of:
tuning an RF coil;
collecting a plurality of signal data from the RF coil;
determining a variance corresponding to the plurality of signal data; and
converting the variance to an absolute temperature data.

6. The method of claim 5, further comprising the steps of:
storing the absolute temperature data; and
steering a field of view of the RF coil.

7. The method of claim 5, further comprising the steps of:
retrieving the stored absolute temperature data; and
displaying an image corresponding to the stored temperature data and the field of view of the RF coil.

8. The method of claim 5, wherein the step of converting the variance to an absolute temperature comprises the step of multiplying the variance by a calibration coefficient.

9. The method of claim 5, further comprising the step of setting a bandwidth, before the step of collecting a plurality of signal data.

10. The method of claim 5, further comprising the step of setting a center frequency before the step of tuning an RF coil.

11. The method of claim 5, wherein the step of determining a variance corresponding to the plurality of signal data comprises the step of histogramming the plurality of signal data.

12. The method of claim 5, further comprising the step of setting a number of samples corresponding to the plurality of signal data.

13. The method of claim 5, wherein the step of collecting a plurality of signal data comprises the step of removing outlier data from the plurality of signal data.

14. A method for calibrating an MR scanner for measuring absolute temperature of a target volume, the method comprising the steps of:
placing a first phantom having a first temperature within a field of view of an RF coil;
tuning the RF coil;
collecting a first plurality of signal data from the RF coil;
determining a first variance corresponding to the first plurality of signal data;
placing a second phantom having a second temperature within the field of view of the RF coil;
collecting a second plurality of signal data from the RF coil;
determining a second variance corresponding to the second plurality of signal data; and
computing a calibration coefficient corresponding to the relation between the first and second temperature and the first and second variance.

15. The method of claim 14, further comprising the step of setting a bandwidth before the step of collecting a first plurality of signal data.

16. The method of claim 16, wherein the calibration coefficient corresponds to the bandwidth.

17. The method of claim 14, further comprising the step of setting a number of samples corresponding to the plurality of signal data before the step of collecting a first plurality of signal data.

18. The method of claim 18, wherein the calibration coefficient corresponds to the number of samples.

19. The method of claim 14, further comprising the step of storing the calibration coefficient after the step of computing the calibration coefficient..

20. A computer readable medium encoded with a program comprising the steps of:
issuing an instruction to tune an RF coil;
collecting a plurality of signal data from the RF coil;
determining a variance corresponding to the plurality of signal data; and
converting the variance to an absolute temperature data.

21. The computer readable medium of claim 21, wherein the program further comprises the step of issuing an instruction to steer a field of view of the RF coil, before the step of collecting a plurality of signal data.

22. The computer readable medium of claim 22, wherein the program further comprises the steps of:
storing the absolute temperature data; and
storing data corresponding to the field of view of the RF coil.

23. The computer readable medium of claim 23, wherein the program further comprises the steps of:
retrieving the absolute temperature data;
retrieving the data corresponding to the field of view of the RF coil; and
constructing an image of the absolute temperature data.+

24. A method for generating thermal imagery of a tissue, the method comprising the steps of:
measuring a magnetic field sensitivity distribution;
estimating an electric field distribution corresponding to the magnetic field sensitivity distribution;
measuring an RF coil impedance;
estimating an electrical conductivity distribution corresponding to the impedance and the electrical field distribution; and
estimating a temperature distribution corresponding to the electrical conductivity distribution and the electric field distribution.
